(19) United States
(12) Patent Application Publication
(10) Pub. No.: US 2016/0192883 A1
(43) Pub. Date: Jul. 7, 2016

(54) OXYGEN SATURATION MEASUREMENTS

(71) Applicant: LifeWatch Technologies, LTD., Rehovot (IL)

(72) Inventors: Michael Pasternak, Bat-Yam (IL); Yossi Lovton, Nes Zeyona (IL)

(21) Appl. No.: 14/590,149

(22) Filed: Jan. 6, 2015

(51) Int. Cl. A61B 5/00; A61B 5/11; A61B 5/01; A61B 5/1455

(52) U.S. Cl. CPC A61B 5/6823; A61B 5/14551; A61B 5/7278; A61B 5/6832; A61B 5/11; A61B 5/721; A61B 5/01; A61B 5/0004; A61B 5/746; A61B 2562/04; A61B 2560/0214

(57) ABSTRACT

A method for measuring oxygen saturation of a user, the method includes illuminating a sternal angle of the user by electromagnetic radiation; and sensing, by an oxygen saturation sensor included in a device that is removably attached to a user, radiation emitted from the sternal angle of the user as a result of the illuminating, wherein the sensing occurs while the oxygen saturation sensor faces the sternal angle of the user; and generating detection signals by the oxygen saturation sensor in response to the sensing of the radiation, wherein the detection signals are indicative of an oxygen saturation characteristic of the user.

### BACKGROUND OF THE INVENTION

Oxygen saturation measurements provide highly valuable information about the state of a user. Results of oxygen saturation measurements depend upon the location of measurement and may be required to be taken over relatively long periods.

There is a growing need to provide methods for accurate oxygen saturation measurements that can be easily taken over long periods of time.

### SUMMARY OF THE INVENTION

According to an embodiment of the invention there may be provided a method that may include illuminating a sternal angle of the user by electromagnetic radiation; and sensing, by an oxygen saturation sensor included in a device that is removably attached to a user, radiation emitted from the sternal angle of the user as a result of the illuminating, wherein the sensing occurs while the oxygen saturation sensor faces the sternal angle of the user; and generating detection signals by the oxygen saturation sensor in response to the sensing of the radiation, wherein the detection signals are indicative of an oxygen saturation characteristic of the user.

The method may include processing the detection signals by the oxygen saturation sensor to provide an indication of the oxygen saturation characteristic of the user. The device may include a sheet having an underside provided with a self-adhesive.

The method may include sensing, by a movement sensor of the device, a movement of the user during the sensing of the radiation; and determining an accuracy of the detection signals in response to movement of the user.

The method may include rejecting detection signals that represent radiation sensed when the user movement exceeds a movement threshold.

The method may include sensing the radiation by a plurality of photodiodes that are coupled in parallel to each other.

The method may include sensing a temperature of the user by a temperature sensor of the device.

The illuminating may include illuminating the sternal angle of the user by a diode that emits visible light pulses and infrared pulses in an interleaved manner.

The illumination may be executed by an illumination module of the device.

The method may include detecting ambient illumination of the sternal angle by processing detection signals generated in response to sensing radiation emitted from the sternal angle at points in time where the sternal angle is not illuminated by the illumination module of the device.

The method may include generating an alert indicative of a detection of the ambient illumination.

The method may include sensing the radiation by a plurality of photodiodes that are arranged in a radially symmetrical manner.

The method may include sending the detection signals to a processor of the device and processing the detection signals by the processor to provide a result indicative of the oxygen saturation characteristic of the user.

The method may include feeding the processor and the oxygen saturation sensor with power from a battery positioned within a lower case of the device; wherein the processor is positioned within an upper case of the device.

The method may include wirelessly transmitting by a transmitter of the device information about the oxygen saturation characteristic of the user.

According to an embodiment of the invention there may be provided a device that is removably attached to a user and may include an oxygen saturation sensor, wherein the oxygen saturation sensor may be configured to: (a) sense radiation emitted from a sternal angle of a user as a result of an illuminating of the sternal angle of the user, wherein the sensing occurs while the oxygen saturation sensor faces the sternal angle of the user; and (b) generate detection signals in response to the sensing of the radiation, wherein the detection signals are indicative of an oxygen saturation characteristic of the user.

The device may be configured to process the detection signals by the oxygen saturation sensor to provide an indication of the oxygen saturation characteristic of the user.

The device may include a sheet having an underside provided with a self-adhesive.

The device may include a movement sensor that may be configured to sense a movement of the user during the sensing of the radiation; and wherein the device may be configured to determine an accuracy of the detection signals in response to movement of the user.

The device may be configured to reject detection signals that represent radiation sensed when the user movement exceeds a movement threshold.

The oxygen saturation sensor may include a plurality of photodiodes that are coupled in parallel to each other.

The device may include a temperature sensor that may be configured to sense a temperature of the user.

The device may be configured to illuminate the sternal angle of the user by a diode that emits visible light pulses and infrared pulses in an interleaved manner.

The device may include an illumination module that may be configured to illuminate the sternum angle of the user.

The device may be configured to detect ambient illumination of the sternal angle by processing detection signals generated in response to sensing radiation emitted from the sternal angle at points in time where the sternal angle is not illuminated by the illumination module of the device.

The device may be configured to generate an alert indicative of a detection of the ambient illumination.

The device may include a plurality of photodiodes that are configured in a radially symmetrical manner.

The device may include a processor that may be configured to receive the detection signals and to process the detection signals by the processor to provide a result indicative of the oxygen saturation characteristic of the user.

The device may include a battery that may be configured to feed the processor and the oxygen saturation sensor with power, wherein the battery is positioned within a lower case of the device; wherein the processor is positioned within an upper case of the device.

The device may include a transmitter that may be configured to wirelessly transmit information about the oxygen saturation characteristic of the user.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof may best be understood by reference to the following detailed description when read with the accompanying drawings in which:

FIG. 1 illustrates the sternum and the ribs of a person;

FIG. 2 is an exploded view of a device according to an embodiment of the invention;

FIG. 3 illustrates a placement of the device of FIG. 2 on a chest of a user according to an embodiment of the invention;

FIG. 4 illustrates a placement of the device of FIG. 2 on a chest of a user according to an embodiment of the invention;

FIG. 5 is a schematic diagram of various components of the device of FIG. 2 according to an embodiment of the invention;

FIG. 6 is a timing diagram according to an embodiment of the invention;

FIG. 7 illustrates a method according to an embodiment of the invention;

FIG. 8 illustrates a method according to an embodiment of the invention; and

FIG. 9 illustrates a method according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It has been surprisingly found that measuring oxygen saturation by illuminating the sternal angle of a user provides reliable results. The sternal angle is easy to find by the user (or third parties) so that users can easily and accurately position the sensor to face sternal angle. This greatly increases the repetitiveness of the oxygen saturation results. Furthermore—placing the device in this position reduces the breath induced movements that the device experiences and further increases the accuracy of this measurement. In addition—placing the device at that position is relatively easy as the sternum is relatively flat.

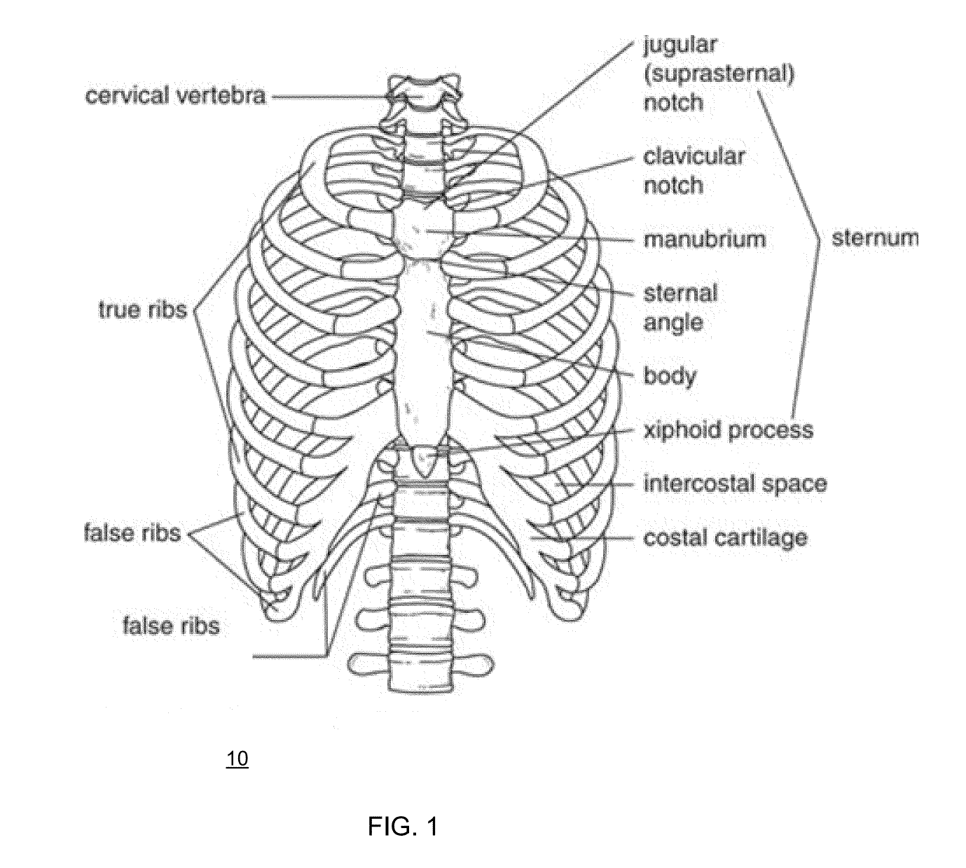

FIG. 1 illustrates the sternum and the ribs of a person 10. The sternum angle is located between the manubrium bone and the body of the sternum.

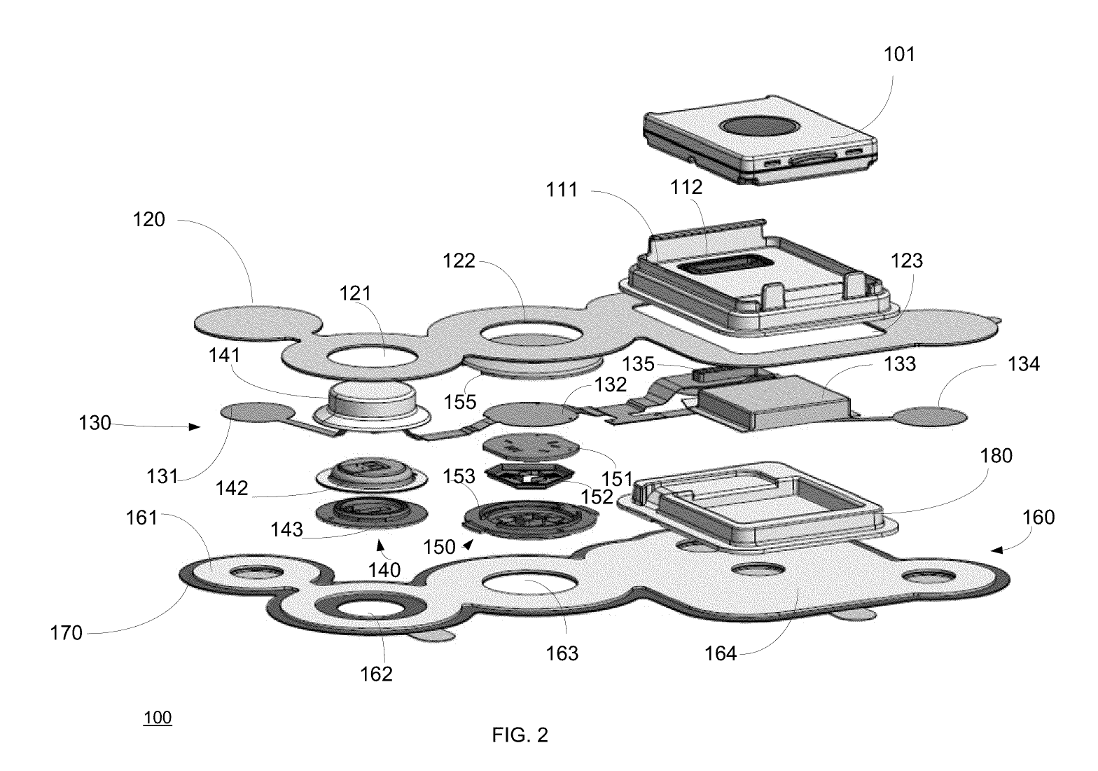

FIG. 2 is an exploded view of a device 100 according to an embodiment of the invention.

Device 100 includes:
1. Processor and transceiver (collectively denoted 101).
2. An upper elastic layer 120 that include first, second and third openings 121, 122 and 123.
3. Intermediate layer 130 that includes conductors 131, 132 and 134 and socket 135 for conveying power from battery 133.
4. Temperature sensor 140 that includes temperature sensor cover 141, temperature sensor electrical board 142 and temperature sensor case 143.
5. Oxygen saturation sensor 150 that includes oxygen saturation sensor electrical board 151, 151, oxygen saturation sensor shield 152 and oxygen saturation sensor case 153.
6. A lower elastic layer 160 that include first, second and third openings 161, 162 and 163 and an addition portion 164 to be contacted by lower case 180. The lower elastic layer 160 has an underside provided with a self-adhesive. Removable cover 170 shields the self-adhesive and is removed before attaching the device 100 to a user.
7. Upper case 111 having socket 112.
8. Lower case 180.

The temperature sensor cover 141 is shaped and positioned to pass through the first opening 121 of the upper elastic layer 120. Cover 155 is arranged to seal the second opening 122 of the upper elastic layer 120. Cover 155 is positioned between the upper elastic layer 120 and conductor 132 of the intermediate layer 120. Conductor 132 is positioned above the oxygen saturation sensor electrical board 151.

The temperature sensor case 143 is positioned directly above the first opening 162 of the lower elastic layer 160.

The oxygen saturation sensor 150 is positioned directly above the second opening 163 of the lower elastic layer 160. It may contact the sternum angle during measurements but may be positioned slightly (few millimeters) above the sternum angle without contacting the sternum angle.

Battery 133 is placed within lower case 180 and its upper facet supports a lower facet of upper case 111 that is connected to the processor and transceiver 101.

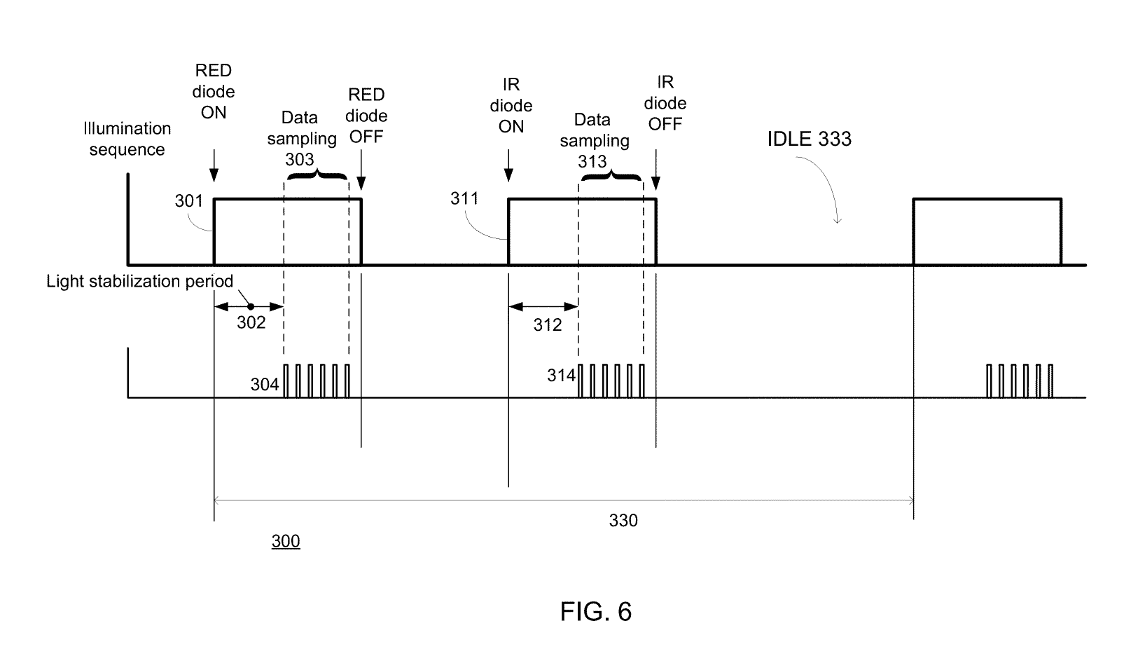

Device 100 is illustrated as including a temperature sensor 140 and oxygen saturation sensor 150. It is noted that other sensor (or sensors) can be provided instead (or in addition) to the temperature sensor 140. Alternatively, the only sensor included in device 100 may be the oxygen saturation sensor 150. For an example (illustrated in FIG. 6), the device 100 may include a movement sensor 144, a temperature sensor 140 and the oxygen saturation sensor 150.

The device 100 may be very compact and light weight. Its transceiver (denoted 101(2) in FIG. 6) may be arranged to perform short range and/or long range transmissions.

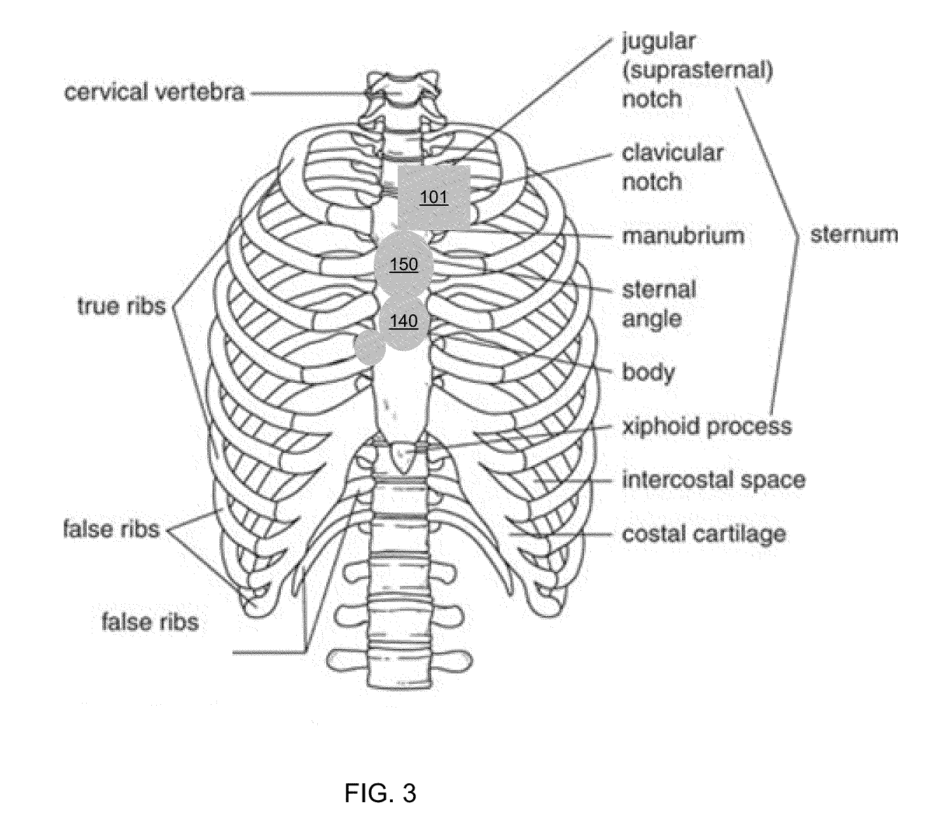

FIG. 3 illustrates device 100 as being positioned on a user wherein the oxygen saturation sensor 150 is positioned directly above the sternum angle, the temperature sensor 140 is positioned below the sternum angle and the processor/transceiver 101 is positioned above the sternum angle.

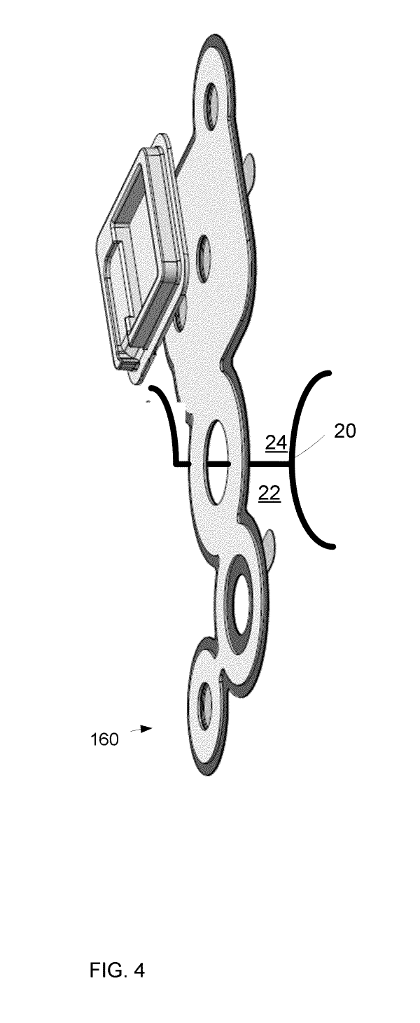

FIG. 4 illustrates the lower elastic layer 160 of device 100 as being positioned on a user wherein the third opening 163 (that the oxygen saturation sensor 150 is positioned directly above) is positioned directly above the sternum angle 22, the temperature sensor 140 is positioned directly above the body 24 of the sternum and the lower case 180 faces the manubrium bone 24.

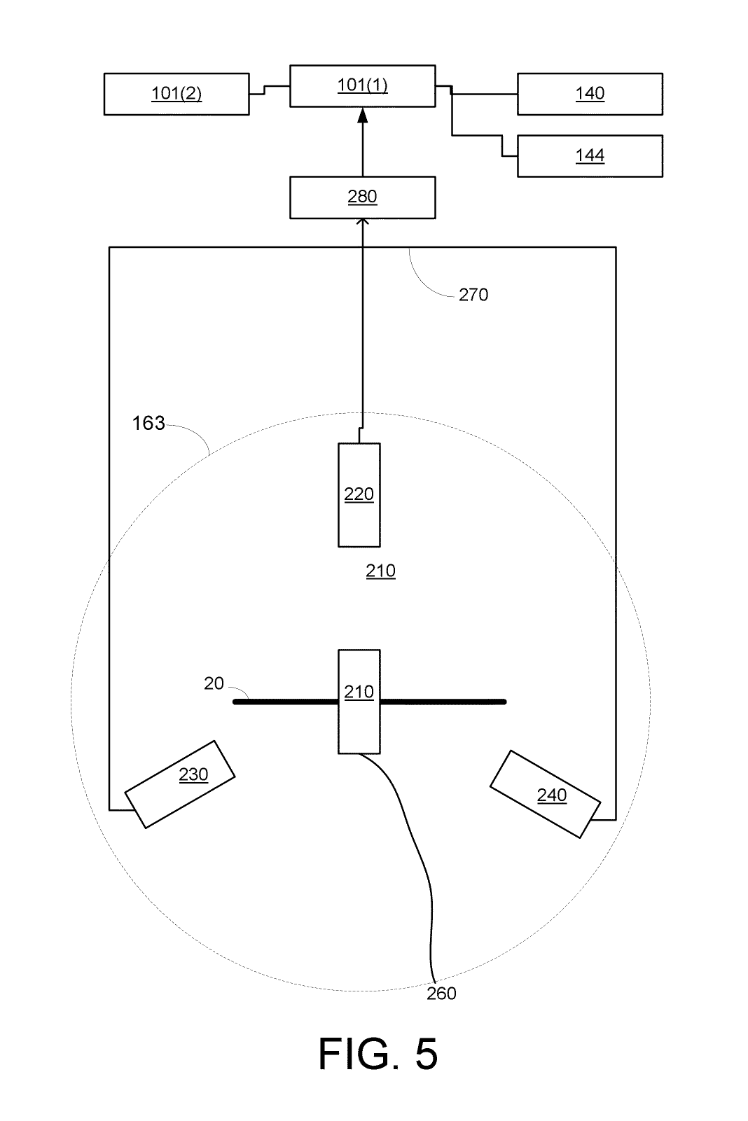

FIG. 5 is a schematic diagram of various components of the device 100 of FIG. 2 according to an embodiment of the invention.

FIG. 5 illustrates the oxygen saturation sensor 150 as including three radiation sensing elements 220, 230 and 240, illumination module 210 (illustrated as being positioned directly above the sternum angle 20 and within third opening 163 of the lower elastic layer 160), intermediate module 260 (that may include an analog amplifier, an analog to digital converter or a combination of both), processor 101(1) of processor/transducer 101, transducer 101(2), temperature sensor 140 and movement sensor 144.

The illumination module 210 may be arranged to illuminate the sternum angle with infrared pulses and visible light pulses. The radiation sensing elements 220, 230 and 240 may sense radiation reflected and/or scattered from the sternum angle in the infrared and visible light ranges and send detection signals towards intermediate module 260.

Pulses of energy are provided to the illumination module 210 via conductor 210.

Radiation sensing elements 220, 230 and 240 are coupled in parallel to each other via conductors 270 but may be coupled in a serial manner to each other.

Processor 101(1) may receive detection signals from temperature sensor 140 and movement sensor 144. It may be arranged to disregard detection signals obtained when the user moves in a manner that may reduce the reliability of the detection signals below a predefined threshold.

FIG. 6 is a timing diagram 300 according to an embodiment of the invention. It illustrates a cyclic illumination pattern having a period of 330. Each cycle includes an activation window 301 of a red diode (delimited between RED diode ON and RED diode OFF) and an activation window 313 of an infrared diode (delimited between IR diode ON and IR diode OFF) that are followed by an idle period 333. Each activation window includes a stabilization period (302 and 312 respectively) in which the emitted light (red or infrared) is stabilized that is followed by a measurement period (303 and 313) in which the light pulses (304 and 314 respectively) can be used for oxygen saturation measurements. The activation windows may be of the same length (for example 0.5 millisecond) or of different lengths. The cyclic illumination pattern may have a cycle 330 that is longer and even much longer than the duration of the activation windows (for example—13 millisecond).

Detection signals generated during idle period 333 may be indicative of unwanted ambient light.

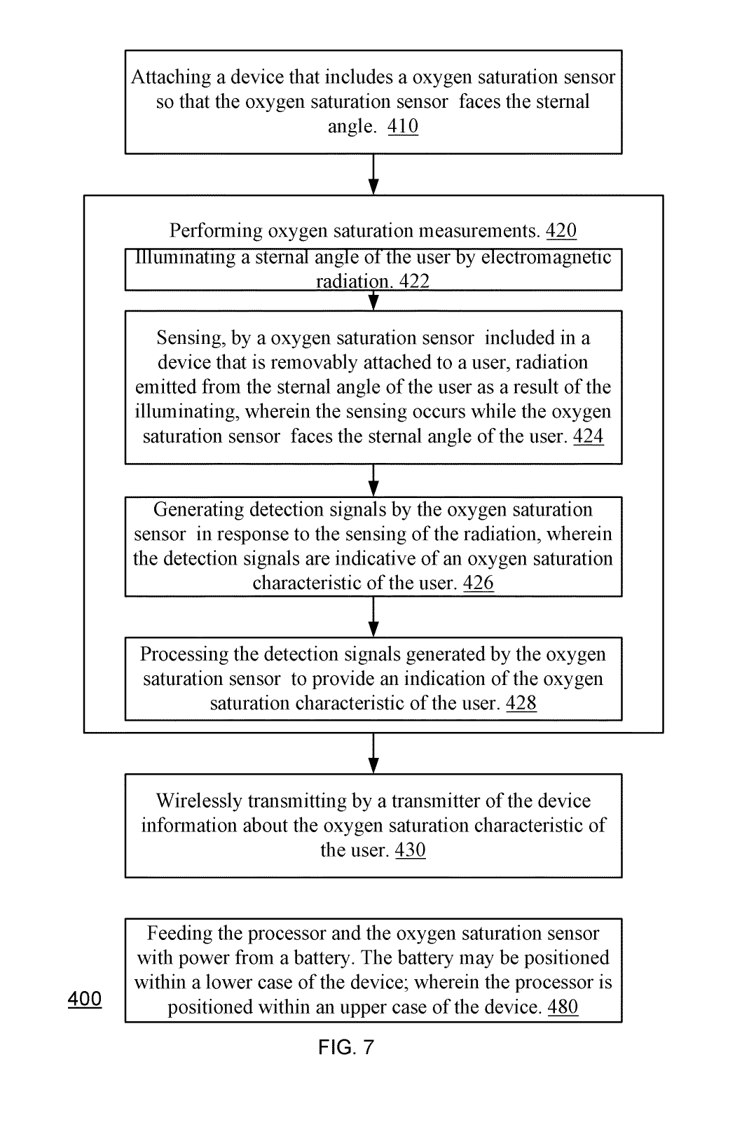

FIG. 7 illustrates method 400 according to an embodiment of the invention.

Method 400 may start by stage 410 of attaching a device that includes an oxygen saturation sensor so that the oxygen saturation sensor faces the sternal angle. This may, for example, positioning device 100 (or any other device that has an oxygen saturation sensor for sensing oxygen saturation characteristics) on a user. The device can be attached using a self-adhesive material, using a belt and the like.

Stage 410 may be followed by stage 420 of performing oxygen saturation measurements. Multiple oxygen saturation measurements can be performed over short or long periods of time—minutes, hours, days and even more.

An oxygen saturation measurement may include a detection signal acquisition phase and a processing phase. The detection signal acquisition phase is executed by the device attached to the client. The processing stage can be executed in full by the device, can be partially executed by the device or can be executed by another device or system not attached to the device.

The detection signal acquisition stage includes:
1. Illuminating (stage 422) a sternal angle of the user by electromagnetic radiation.
2. Sensing (stage 424) by an oxygen saturation sensor included in a device that is removably attached to a user, radiation emitted from the sternal angle of the user. The radiation detected can result from the illuminating of the sternal angle. The sensing occurs while the oxygen saturation sensor faces the sternal angle of the user.
3. Generating detection signals (stage 426) by the oxygen saturation sensor in response to the sensing of the radiation, wherein the detection signals are indicative of an oxygen saturation characteristic of the user.

Stage 422 may include illuminating the sternal angle of the user by a diode that emits visible light pulses and infrared pulses in an interleaved manner.

Stage 422 may be executed by an illumination module of the device.

Stage 424 may include sensing the radiation by one or more sensing elements such as photodiodes. If there are multiple sensing elements the sensing elements may be coupled to each other in parallel, in serial or a combination thereof.

Stage 424 may include sensing the radiation by a plurality of photodiodes that are arranged in a radially symmetrical manner.

The processing phase includes processing (stage 428) the detection signals generated by the oxygen saturation sensor to provide an indication of the oxygen saturation characteristic of the user.

If the processing is performed by a processor of the device then stage 428 is preceded (or includes) sending the detection signals to the processor of the device. If the processing is executed by a processor that does not belong to the device then the method includes transmitting the detection signals towards that processor.

Stage 420 may be followed by stage 430 of wirelessly transmitting by a transmitter of the device information about the oxygen saturation characteristic of the user.

Method 400 may also include stage 480 of feeding the processor and the oxygen saturation sensor with power from a battery. The battery may be positioned within a lower case of the device. The processor may be positioned within an upper case of the device.

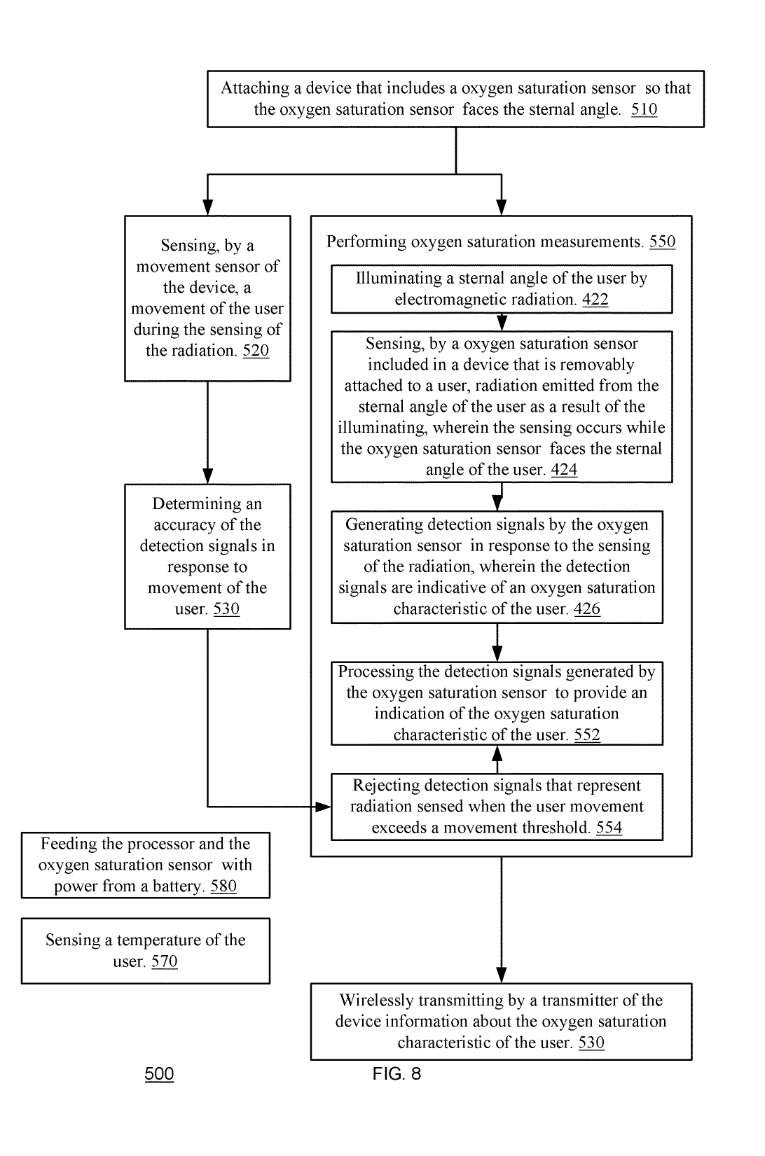

FIG. 8 illustrates method 500 according to an embodiment of the invention.

Method 500 starts by stage 510 of attaching a device that includes an oxygen saturation sensor so that the oxygen saturation sensor faces the sternal angle.

Stage 510 may be followed by stages 520 and 550.

Stage 520 may include sensing, by a movement sensor of the device, a movement of the user during the sensing of the radiation.

Stage 520 may be followed by stage 530 of determining an accuracy of the detection signals in response to movement of the user.

Stage 550 may include of performing oxygen saturation measurements. Multiple oxygen saturation measurements can be performed over short or long periods of time-minutes, hours, days and even more.

Stage 550 may include stages 422, 424 and 426. Stage 550 may also include stage 552 of processing the detection signals by the oxygen saturation sensor to provide an indication of the oxygen saturation characteristic of the user and stage 554 of rejecting detection signals that represent radiation sensed when the user movement exceeds a movement threshold.

If the processing is performed by a processor of the device then stage 552 is preceded (or includes) sending the detection signals to the processor of the device. If the processing is executed by a processor that does not belong to the device then the method includes transmitting the detection signals towards that processor.

Stage 550 may be followed by stage 560 of wirelessly transmitting by a transmitter of the device information about the oxygen saturation characteristic of the user.

Method 500 may also include stage 580 of feeding the processor and the oxygen saturation sensor with power from a battery. The battery may be positioned within a lower case of the device. The processor may be positioned within an upper case of the device.

FIG. 8 also illustrates method 500 as sensing (570) a temperature of the user by a temperature sensor of the device. It is noted that this stage can include performing any further sensing operation by any other type of sensor.

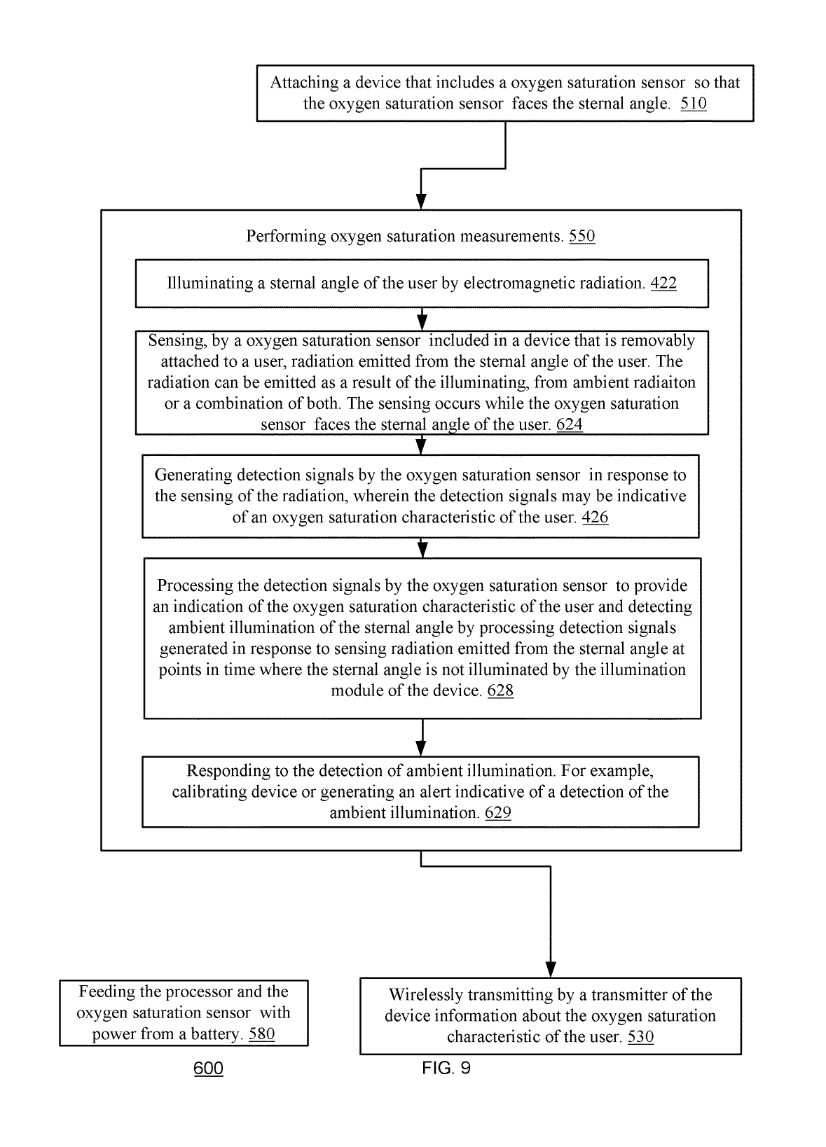

FIG. 9 illustrates method 600 according to an embodiment of the invention.

Method 600 may start by stage 610 of attaching a device that includes an oxygen saturation sensor so that the oxygen saturation sensor faces the sternal angle.

Stage 610 may be followed by stage 620 of performing oxygen saturation measurements.

An oxygen saturation measurement may include a detection signal acquisition phase and a processing phase. The detection signal acquisition phase is executed by the device attached to the client. The processing stage can be executed in full by the device, can be partially executed by the device or can be executed by another device or system not attached to the device.

The detection signal acquisition stage includes:
1. Illuminating (stage 422) a sternal angle of the user by electromagnetic radiation.
2. Sensing (stage 624), by an oxygen saturation sensor included in a device that is removably attached to a user, radiation emitted from the sternal angle of the user. The radiation detected can result of the illuminating of the sternal angle, from ambient illumination of from a combination thereof. The sensing occurs while the oxygen saturation sensor faces the sternal angle of the user.
3. Generating detection signals (stage 426) by the oxygen saturation sensor in response to the sensing of the radiation, wherein the detection signals are indicative of an oxygen saturation characteristic of the user.

Stage 424 may include sensing the radiation by one or more sensing elements such as photodiodes. If there are multiple sensing elements the sensing elements may be coupled to each other in parallel, in serial or a combination thereof.

The processing phase includes processing (stage 628) the detection signals by the oxygen saturation sensor to provide an indication of the oxygen saturation characteristic of the user.

Stage 628 may include detecting ambient illumination of the sternal angle by processing detection signals generated (during stage 426) in response to sensing radiation emitted from the sternal angle at points in time where the sternal angle is not illuminated by the illumination module of the device. See, for example, generation of detection signals that sense ambient radiation sensed during idle period 330 of FIG. 5.

Stage 628 may be followed by stage 629 of responding to the detection of ambient illumination.

For example, calibrating device or generating an alert indicative of a detection of the ambient illumination. The calibrating may include estimating the ambient light and compensating the oxygen saturation measurements in response to the ambient light. For example- reducing from detected radiation (detected when illuminating the sternum angle by IR or light pulse) the estimated value of the ambient light (IR component or light component respectively).

The alert may signal the user that he should re-attach the device in order to reduce or eliminate ambient radiation from reaching the sternum angle.

If the processing is performed by a processor of the device then stage 628 is preceded (or includes) sending the detection signals to the processor of the device. If the processing is executed by a processor that does not belong to the device then the method includes transmitting the detection signals towards that processor.

Stage 620 may be followed by stage 630 of wirelessly transmitting by a transmitter of the device information about the oxygen saturation characteristic of the user.

Method 600 may also include stage 680 of feeding the processor and the oxygen saturation sensor with power from a battery. The battery may be positioned within a lower case of the device. The processor may be positioned within an upper case of the device.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for measuring oxygen saturation of a user, the method comprises:
illuminating a sternal angle of the user by electromagnetic radiation; and
sensing, by an oxygen saturation sensor included in a device that is removably attached to a user, radiation emitted from the sternal angle of the user as a result of the illuminating, wherein the sensing occurs while the oxygen saturation sensor faces the sternal angle of the user; and
generating detection signals by the oxygen saturation sensor in response to the sensing of the radiation, wherein the detection signals are indicative of an oxygen saturation characteristic of the user.

2. The method according to claim 1 comprises processing the detection signals by the oxygen saturation sensor to provide an indication of the oxygen saturation characteristic of the user.

3. The method according to claim 1 wherein the device comprises a sheet having an underside provided with a self-adhesive.

4. The method according to claim 1, comprising sensing, by a movement sensor of the device, a movement of the user during the sensing of the radiation; and determining an accuracy of the detection signals in response to movement of the user.

5. The method according to claim 4 comprising rejecting detection signals that represent radiation sensed when the user movement exceeds a movement threshold.

6. The method according to claim 1 comprising sensing the radiation by a plurality of photodiodes that are coupled in parallel to each other.

7. The method according to claim 1 further comprising sensing a temperature of the user by a temperature sensor of the device.

8. The method according to claim 1 wherein the illuminating comprises illuminating the sternal angle of the user by a diode that emits visible light pulses and infrared pulses in an interleaved manner.

9. The method according to claim 1 wherein the illumination is executed by an illumination module of the device.

10. The method according to claim 9 comprising detecting ambient illumination of the sternal angle by processing detection signals generated in response to sensing radiation emitted from the sternal angle at points in time where the sternal angle is not illuminated by the illumination module of the device.

11. The method according to claim 10 comprising generating an alert indicative of a detection of the ambient illumination.

12. The method according to claim 1 comprising sensing the radiation by a plurality of photodiodes that are arranged in a radially symmetrical manner.

13. The method according to claim 1 further comprising sending the detection signals to a processor of the device and processing the detection signals by the processor to provide a result indicative of the oxygen saturation characteristic of the user.

14. The method according to claim 13 comprising feeding the processor and the oxygen saturation sensor with power from a battery positioned within a lower case of the device; wherein the processor is positioned within an upper case of the device.

15. The method according to claim 13 further comprising wirelessly transmitting by a transmitter of the device information about the oxygen saturation characteristic of the user.

16. A device that is removably attached to a user and comprises an oxygen saturation sensor, wherein the oxygen saturation sensor is configured to: (a) sense radiation emitted from a sternal angle of a user as a result of an illuminating of the sternal angle of the user, wherein the sensing occurs while the oxygen saturation sensor faces the sternal angle of the user; and (b) generate detection signals in response to the sensing of the radiation, wherein the detection signals are indicative of an oxygen saturation characteristic of the user.

17. The device according to claim 16 wherein the device is configured to process the detection signals by the oxygen saturation sensor to provide an indication of the oxygen saturation characteristic of the user.

18. The device according to claim 16 wherein the device comprises a sheet having an underside provided with a self-adhesive.

19. The device according to claim 1, comprising a movement sensor that is configured to sense a movement of the user during the sensing of the radiation; and wherein the device is configured to determine an accuracy of the detection signals in response to movement of the user.

20. The device according to claim 19 wherein the device is configured to reject detection signals that represent radiation sensed when the user movement exceeds a movement threshold.

21. The device according to claim 16 wherein the oxygen saturation sensor comprises a plurality of photodiodes that are coupled in parallel to each other.

22. The device according to claim 16 comprising a temperature sensor that is configured to sense a temperature of the user.

23. The device according to claim 16 wherein the device is configured to illuminate the sternal angle of the user by a diode that emits visible light pulses and infrared pulses in an interleaved manner.

24. The device according to claim 16 wherein the device comprises an illumination module that is configured to illuminate the sternum angle of the user.

25. The device according to claim 24 wherein the device is configured to detect ambient illumination of the sternal angle by processing detection signals generated in response to sensing radiation emitted from the sternal angle at points in time where the sternal angle is not illuminated by the illumination module of the device.

26. The device according to claim 25 wherein the device is configured to generate an alert indicative of a detection of the ambient illumination.

27. The device according to claim 16 comprising a plurality of photodiodes that are configured in a radially symmetrical manner.

28. The device according to claim 16 comprising a processor that is configured to receive the detection signals and to process the detection signals by the processor to provide a result indicative of the oxygen saturation characteristic of the user.

29. The device according to claim 28 comprising a battery that is configured to feed the processor and the oxygen saturation sensor with power, wherein the battery is positioned within a lower case of the device; wherein the processor is positioned within an upper case of the device.

30. The device according to claim 28 comprising a transmitter that is configured to wirelessly transmit information about the oxygen saturation characteristic of the user.
